(19) United States
(12) Patent Application Publication
(10) Pub. No.: US 2005/0276720 A1
(43) Pub. Date: Dec. 15, 2005

(54) System and method for providing germicidal lighting for poultry facilities

(76) Inventor: Rafael S. Correa, Salisbury, MD (US)

Correspondence Address: JACOBSON HOLMAN PLLC, 400 SEVENTH STREET N.W., SUITE 600, WASHINGTON, DC, 20004, US

(21) Appl. No.: 11/141,190

(22) Filed: Jun. 1, 2005

Related U.S. Application Data
(60) Provisional application No. 60/575,410, filed on Jun. 1, 2004.

(51) Int. Cl. A61L 2/00

(52) U.S. Cl. 422/24

(57) ABSTRACT

The present invention provides a system and method to reduce or eliminate bacteria and fungus on surfaces, equipment or vehicles in poultry processing facilities without the use of antibiotics or antiseptics. The invention provides a cycling system and method which utilize ultraviolet light of a preferred wavelength (UV-C) to reduce or eliminate bacteria and fungus on surfaces, equipment or vehicles in poultry processing facilities and which can be used in poultry processing facilities without harm to the poultry or workers.

This application claims the benefit of priority to U.S. provisional patent application Ser. No. 60/575,410, filed Jun. 1, 2004.

### FIELD OF INVENTION

The present invention relates to the use of ultra-violet (UV) light at specific germicidal wavelengths to sterilize a poultry house, hatchery or processing facility to prevent infection. Use of UV to reduce or eliminate surface and airborne bacteria greatly reduces the need for antibiotics and other chemical sterilization methods to keep poultry infection free. The system is especially useful in poultry production and processing areas such as hatching cabinets, throwing rooms, and controlled environment poultry rearing rooms.

### INTRODUCTION

The environment of newly hatched poultry quickly becomes contaminated with microorganisms as soon as the actual hatching process or exit from the eggs begins. The microorganisms include, but are not limited to, (1) bacteria such as Salmonella species, Escherichia coli, staphylococcus aureus and (2) fungal organisms such as members of fungal genus Aspergillus, and possibly avian Mycoplasmas. This rapid rise in the concentration of microorganisms is often referred to as a bacterial bloom which follows the pipping stage of incubation in poultry. It is at this stage that optimum conditions for growth of microorganisms exist in terms of humidity, temperature, and nutrient levels. Organic debris present from the hatching process provides abundant levels of nutrients enhancing microbial replication at this time.

The microbial levels in this environment are commonly measured by microbiological culture of air or by measurement of microbial levels contained on specific quantities of hatcher down or fluff (a by product of the bird produced during hatch).

The consumption of improperly prepared poultry products has resulted in numerous cases of human intestinal diseases. It has long been recognized that Salmonella are causative agents of such diseases, and more recently Campylobacter. As many as two million cases of salmonellosis occur annually in the United States (Stavrix et al., Journal of Food Protection, Volume 56, No. 2, 173-180, February, 1993); twice as many cases of campylobacteriosis are thought to occur (Krienberg et al., Food Technology, pages 77-81, 98, July 1987). Both microorganisms may colonize poultry gastrointestinal tracts without any deleterious effects on the birds, and although some colonized birds can be detected, asymptomatic carriers can freely spread the microorganisms during production and processing, resulting in further contamination of both live birds and carcasses. Poultry serve as the primary reservoir for Salmonella and Campylobacter in the food supply (Jones et al., Journal of Food Protection, Volume 54, No. 4, 259-262, April 1991; Jones et al., Journal of Food Protection, Volume 54, No. 7, 502-507, July 1991). The intestinal contents of chickens may harbor up to 107 Campylobacter and/or Salmonella organisms per gram, and cross contamination during processing is frequent (Oosterom et al., Journal of Food Protection, Volume 46, No. 4, 339-344, April 1983). Studies have demonstrated that fecal material constitutes the major source from which edible parts of chickens are contaminated in processing plants. Therefore, to significantly reduce the level of contamination on processed poultry, pathogen-free birds must be delivered to the processing plant (Bailey, Poultry Science, Volume 72, 1169-1173, 1993).

Better control measures are needed to minimize the spread of these and other human enteropathogenic bacteria; and the most promising approach to achieve this end has been to decrease the incidence and level of colonization by these microorganisms in poultry gastrointestinal tracts. Hatching cabinets are known to be one of the primary sources for human enteropathogenic bacteria contamination of poultry. A considerable amount of dust is generated during the hatching process from the time of piping on day 20 through final hatching on day 21 of incubation. The dust is caused by the breaking up of egg shells and feather particles which are entrained into the air from the new chicks as they move around. Bailey et al (Poultry Science, Volume 71 (1):6; and Poultry Science, Volume 73(7), 1153-1157, 1994) demonstrated that a single salmonella contaminated egg could contaminate most of the eggs and newly hatched chicks in a hatching cabinet. This result suggests that extensive airborne transmission of the bacteria is possible since the typical hatching cabinet has several trays of fertile eggs on several different levels and on several different carts. Eggshell fragments, belting materials, and paper pads used in commercial hatcheries have also been shown to be sources of salmonella contamination (Cox et al, Poultry Sciences, Volume 69, 1606-1609, 1990).

Various intervention approaches have been taken in attempts to reduce airborne transmission of disease. Bailey et al (Poultry Science, Volume 75(2), 191-196, 1996) have demonstrated that chemical treatment of hatching cabinet air between day 18 and hatch can significantly reduce disease transmission caused by eggs which are internally contaminated with Salmonella. Hydrogen peroxide treatment was the most effective in reducing salmonella on the shells, in the air, and in the chicks. Hopkins and Drury (Avian Diseases, Volume 15, 596-603, 1971) have demonstrated the ability of airborne diseases such as Newcastle disease virus (NDV) to be transmitted from groups of donor chickens to groups of susceptible chickens and the ability of high efficiency filters to interrupt this transmission.

Saurenman et al. (U.S. Pat. No. 3,696,791) disclose the use of air ionization for reducing air-borne particulates and ammonia gas and odors in feeding enclosures of animals, especially poultry. Ions are dispersed into the feeding areas. The device includes a means to disperse ions suspended overhead, a static voltage sensor, a control device, a voltage generator, a blower, and a positively charged conductive grid.

Another means of sterilization that has been used is the washing down or fumigation of facilities using heavily chlorinated water to wash all surfaces, equipment or vehicles. However, use of this or similar methods results in severe corrosion of all metallic surfaces, so that this method is not favored for ordinary use.

Ultraviolet (UV) light has been long used for disinfection and sterilization. Ultraviolet light may be produced artificially by electric-arc lamps. Recently, the widespread availability of low to medium pressure mercury bulbs has led to the development of devices which use UV-C to decontaminate water supplies. UV-C is a high frequency wavelength of light within the ultraviolet band and has been shown to be the most bactericidal type of ultraviolet light. UV-C has wavelengths of about 280 nm to 150 nm. The only recent availability of the appropriate bulbs as well as significant safety concerns regarding worker exposure to UV-C likely contribute to the lack of efforts to use UV-C outside of self-contained water purification systems.

### OBJECTS OF THE INVENTION

It is an object of the present invention to provide a system to reduce or eliminate bacteria and fungus on surfaces, equipment or vehicles in poultry processing facilities without the use of antibiotics or antiseptics.

It is another object of the present invention to provide a system which utilizes ultraviolet (UV) light to reduce or eliminate bacteria and fungus on surfaces, equipment or vehicles in poultry processing facilities.

It is a further object of the invention to provide a UV light sterilization system that can be used in poultry processing facilities without harm to the poultry or workers.

### BRIEF DESCRIPTION OF THE DRAWINGS

FIG. 1 shows a schematic diagram of one embodiment of the germicidal lighting system.

FIG. 2 shows a diagram of placements in an embodiment of the present invention.

### DETAILED DESCRIPTION AND PREFERRED EMBODIMENTS

In describing embodiments of the invention, specific terminology will be resorted to for the sake of clarity. However, the invention is not intended to be limited to the specific terms so selected, and it is to be understood that each specific term includes all technical equivalents which operate in a similar manner to accomplish a similar purpose.

It is well known that light in the ultraviolet (UV) spectrum can damage DNA of microscopic organisms such as bacteria, fungus and viruses. The wavelengths of light designated as UV light range from approximately 400 nm (long-wave) down to 100 nm (short-wave). The wavelengths that are germicidal, i.e., that damage DNA thereby killing microorganisms, span from about 300 nm to about 100 nm. It is preferable for most germicidal uses to use UV light in the range of about 280 nm to 100 nm (UV-C). UV-C deactivates the DNA of bacteria, viruses and other pathogens and thus destroys their ability to multiply and cause disease. Specifically, UV-C light causes damage to the nucleic acid of microorganisms by forming covalent bonds between certain adjacent bases in the DNA. The formation of such bonds prevents the DNA from being unzipped for replication, and the organism is unable to reproduce.

Examples of pathogenic organisms that are killed by this treatment are bacteria, such as: Agrobacterium lumefaciens 5; Bacillus subtilis; Salmonella enteritidis; Salmonella paratyphi; Clostridium botulinum; Shigella dysenteriae; and Vibrio comma; molds such as: Aspergillus amstelodami; Aspergillus flavus; and Penicillium chrysogenum; protozoa, such as: Chlorella vulgaris; Giardia lamblia; and Paramecium; viruses such as: Adenovirus Type III; Influenza; Coxsackie; Infectious Hepatitis; and Rotavirus; and yeasts such as: Saccharomyces cerevisiae and Baker's Yeast.

There have been a number of studies done on the amount of UV light exposure necessary to kill microorganisms. See, “The Use of Ultraviolet Light for Microbial Control”, Ultrapure Water, April 1989; William V. Collentro, “Treatment of Water with Ultraviolet Light—Part I”, Ultrapure Water, July/August 1986; James E. Cruver, Ph.D., “Spotlight on Ultraviolet Disinfection”, Water Technology, June 1984; Dr. Robert W. Legan, “Alternative Disinfection Methods—A Comparison of UV and Ozone”, Industrial Water Engineering, Mar/Apr 1982; Rudolph Nagy, Research Report BL-R-6-1059-3023-1, Westinghouse Electric Corporation; Myron Lupal, “UV Offers Reliable Disinfection”, Water Conditioning & Purification, November 1993; John Treij, “Ultraviolet Technology”, Water Conditioning & Purification, December 1995; Bak Srikanth, “The Basic Benefits of Ultraviolet Technology”, Water Conditioning & Purification, December 1995; all hereby incorporated by reference into the specification as if fully set forth in their entireties.

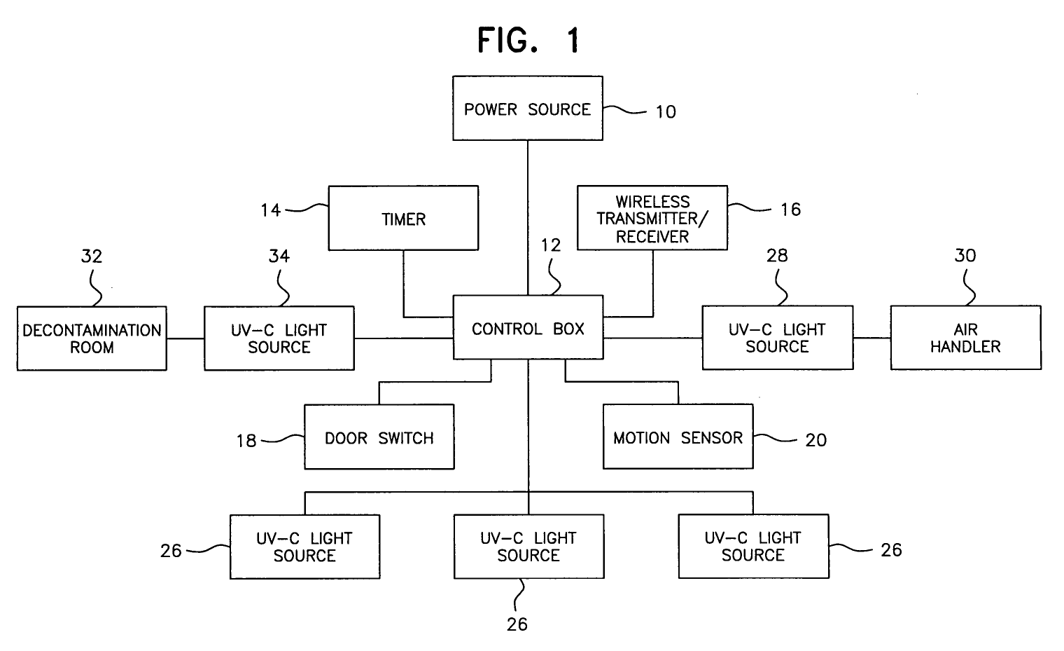

Referring to the schematic in FIG. 1, the UV-C light sources 16 will be connected to a control box 11. The control box 11 contains the circuits, a connection to a power source 10, a timing mechanism 15 and connections to a plurality of door switches 13. It will be necessary to control the UV light sources 16 so as not to expose people accidentally during an exposure cycle. Control of the light sources is done through at least two different mechanisms.

A first mechanism is through a timer system 15. The timer 15 can be mechanical or electronic. In a preferred embodiment, the timer 15 will cycle the UV light source 16 on for about 10 seconds at least once every hour. This intermittent irradiation will be continuous while the chicken house is in use. The period or time of irradiation will be short because of the effectiveness of the UV light exposure over a short period of time, such as about 10 seconds, and otherwise to prevent damage to the eyes of the chickens from unnecessary UV light exposure. In a more preferred embodiment the timer 15 will cycle the UV light source 16 on for about 10 seconds every 7-15 minutes, and most preferably every 10 minutes. The timing cycle is dependant on the efficacy of the exposure time in killing the desired microorganisms and on limiting exposure to the chickens.

A second mechanism for operation of the UV-C lighting system is a manual control which will allow the UV lights 16 to be turned on by an authorized operator. There is contemplated the use of a key or access code so that the light sources 16 could only be operated by authorized personnel. Additionally, the control box or panel can be connected to door switches 13 and motion detectors 14. The purpose of the door switches 13 is to deactivate the UV light sources 16 if a person enters the facility while the UV light source 16 is cycling on. There can also be a cycle delaying mechanism that will not turn the cycle back on until some time interval after the doors are closed. The motion detectors 14 can also be connected to the control box 11 so that the light cycle will not be activated while people are servicing the facility. Examples of lighting system control boxes connected to alarms and motion detectors are known, see for example, U.S. Pat. Nos. 6,309,090 and 5,867,099, which are hereby incorporated by reference into the specificationas if fully set forth in their entireties.

The types of UV light sources 16 useful in the present invention can vary. In a preferred embodiment, lamps using short wave low pressure mercury vapor tubes that produce ultraviolet wavelengths in the UV-C range are used. Examples of such lamps are the Ster-L-Ray™ germicidal ultraviolet lamps manufactured by Atlantic Ultraviolet Corporation of Hauppauge, N.Y.

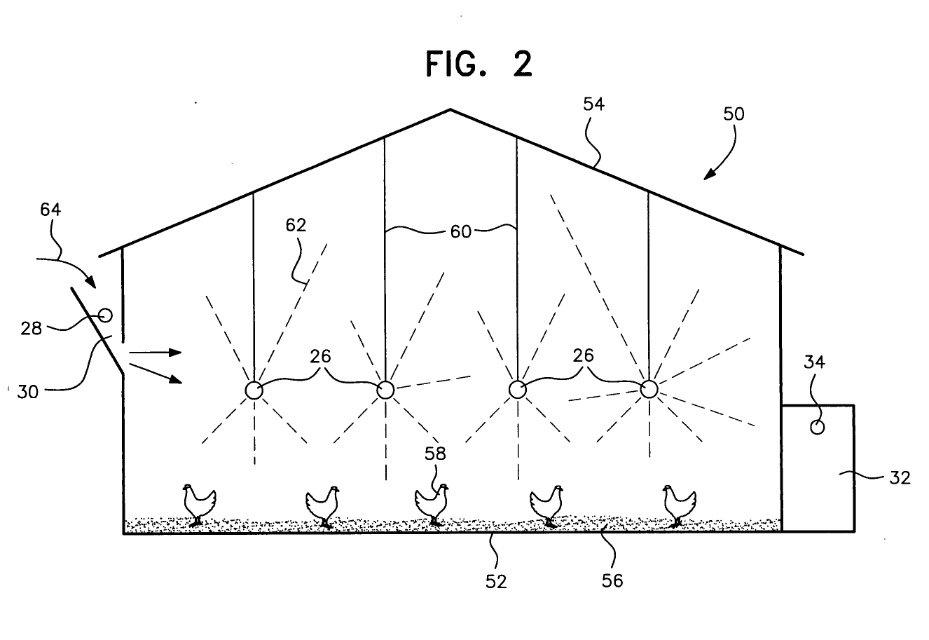

It is contemplated that in a preferred embodiment this system will be used in the large barn type facility known as a chicken house. In commercial use these facilities are large enclosed spaces with low roofs usually less than 10 feet high. Inside, the chickens are stacked in nesting structures. FIG. 2 is a schematic view showing possible placement of UV-C light sources 16 in the present invention. Inside the chicken house 18, are nesting racks 17 where the chickens are placed. Overhead UV-C lights 16 are placed for wide area exposure. UV-C lights 16 can also be placed on or near floor level to kill bacteria that are not exposed to the ceiling UV-C light sources 16.

The chicken houses are periodically emptied and cleaned after a batch of chickens has reached harvesting size. It is also contemplated that during the interval that the chicken houses are empty, the UV light sources 16 can be in a “full sterilization mode” where the UV light sources 16 remain on continuously, until the next batch of chicks is brought in. In this mode, the motion sensors could also be used to detect movement of chickens so as not to engage the sterilization cycle accidentally.

The UV-C light sources 16 will be placed in a variety of locations. Typically, there would be ceiling mounted fixtures for wide area applications. Other smaller UV-C light sources could be mounted near the floor and in other locations so that all surfaces would be exposed to the UV-C light. Locations of UV-C light sources 16 should be determined by geography and geometry of the house, incubator, hatcher or other area to be exposed. The UV-C light sources can be hardwired to a control box or panel, or can be stand-alone devices with their own power source and attached to a wireless or radio-controlled receiver that communicates with a central control transmitter. In an alternative embodiment, the control box can communicate with a computer either hardwired or remotely through a wireless connection. The computer contains software for controlling the timing cycling of the lights and can control any number of facilities.

In another embodiment, the present invention can be used in chicken hatchers and incubators. Chicken hatchers are enclosed cabinet structures that can hold many trays of fertilized chicken eggs. The UV light sources can be internal (positioned inside the hatchers) and turn on periodically as in the chicken house. It is also contemplated that the egg trays used in the incubators and hatchers can be exposed to UV light by means of a conveyor system. For example, when the eggs are hatched the chicks are removed from the hatcher and the trays are then washed normally and placed on a conveyor that would comprise a tunnel from the washing facility to the clean side of the incubating and/or hatching facility. The tunnel can have doors at both ends and within the tunnel a conveyor belt or similar apparatus. Also within the tunnel UV light sources are placed to expose the trays to the UV light for a sufficient time to sterilize them. This method and system could be used on a larger scale for many other types of equipment used in poultry farming.

In another embodiment of the present invention a large enclosed light tight chamber or room has the UV system in place where equipment or vehicles can be placed. Using the same type of system the equipment or vehicles can be exposed to the sterilizing light.

In a further embodiment of the present invention a enclosed light tight booth is made so that workers can be exposed to the light when they enter and exit any of the facilities. Presently, many of the processing plants require workers to shower in and wear special sterile clothing so as to prevent contamination of the livestock. It is contemplated that in addition to, or in place of showering in, the workers could wear safety goggles and receive a short exposure of UV of a sufficient interval to be germicidal.

In another embodiment of the present invention, the system can be adapted to sterilize the air handling systems of the related facilities. For example, UV light sources could be placed inside evaporators and blowers that are used to move air through the chicken house. The lights could be cycled or kept on indefinitely as there would not be a risk of human or livestock exposure to the light inside the apparatus or its ducting.

Having described the invention, many modifications thereto will become apparent to those skilled in the art to which it pertains without deviation from the spirit of the invention as defined by the scope of the appended claims. The disclosures of U.S. Patents, patent applications, and all other references cited above are all hereby incorporated by reference into this specification as if fully set forth in its entirety.

1. A system for reducing or eliminating microbes on surfaces, equipment or vehicles in poultry processing facilities without harm to the poultry or workers comprising:
a source of germicidal ultraviolet radiation disposed to emit said radiation within said poultry processing facility;
a control unit for activating said source of germicidal ultraviolet radiation;
at least one switch means disposed in at least one door, said switch means activated upon opening of said door;
said control unit responsive to activation of said switch means to deactivate said source of germicidal ultraviolet radiation while said door remains open; and
said control unit also having a timer means to activate said source of germicidal ultraviolet radiation for a determined time interval.

2. The system for reducing or eliminating microbes on surfaces, equipment or vehicles in poultry processing facilities of claim 1, wherein said control unit further comprises
at least one motion detection unit wherein activation of said motion detector unit will delay or deactivate aid source of germicidal ultraviolet radiation until there is no further activation of said motion detector unit.

3. The system for reducing or eliminating microbes on surfaces, equipment or vehicles in poultry processing facilities of claim 2, wherein said control unit further comprises
a manual control having an ability to limit activation to authorized users through a key or alphanumerical pad and access code; and
a wireless control unit having the ability to receive or transmit signals from a remote control unit, wherein said wireless control unit can adjust the timing intervals of the control unit through operator commands entered into said remote control unit.

4. The system for reducing or eliminating microbes on surfaces, equipment or vehicles in poultry processing facilities of claim 1, wherein said source of germicidal ultraviolet radiation emits radiation at a range of wavelengths between about 100 nm to 280 nm.

5. The system for reducing or eliminating microbes on surfaces, equipment or vehicles in poultry processing facilities of claim 4, wherein said source of germicidal ultraviolet radiation comprises short wave low pressure mercury vapor tubes or lamps.

6. The system for reducing or eliminating microbes on surfaces, equipment or vehicles in poultry processing facilities of claim 1 wherein the location or placement of germicidal ultraviolet radiation sources can be on nesting racks, overhead or ceiling locations, and on or near floor level.

7. The system for reducing or eliminating microbes on surfaces, equipment or vehicles in poultry processing facilities of claim 3, wherein said control unit can be manually controlled such that the germicidal ultraviolet radiation sources can be activated for an indefinite period of time.

8. The system for reducing or eliminating microbes on surfaces, equipment or vehicles in poultry processing facilities of claim 1 wherein the poultry processing facilities further comprise chicken hatchers and incubators.

9. A system for reducing or eliminating microbes on surfaces of egg trays used in hatchers and incubators comprising:
a washing facility;
an incubation or hatching facility;
a conveyor system for moving the egg trays from the washing facility to the incubation or hatching facility;
wherein said conveyor system connects said washing facility to said incubation or hatching facility and said conveyor system is enclosed in a tunnel like structure;
a source of germicidal ultraviolet radiation disposed to emit said radiation within said tunnel like structure having doors at both ends;
a control unit for activating said source of germicidal ultraviolet radiation;
at least one switch means disposed in at least one door, said switch means activated upon opening of said door;
said control unit responsive to activation of said switch means to deactivate said source of germicidal ultraviolet radiation while said door remains open; and
wherein said conveyor moves the egg trays at a rate which allows sufficient time for the germicidal ultraviolet radiation to sterilize the egg trays.

10. A method for reducing or eliminating microbes on surfaces, equipment or vehicles in poultry processing facilities without harm to the poultry or workers comprising:
providing a source of germicidal ultraviolet radiation disposed to emit said radiation within said poultry processing facility;
controlling the activation of said source of germicidal ultraviolet radiation through the operation of a control unit;
providing at least one switch means disposed in at least one door, said switch means activated upon opening of said door;
said control unit responding to activation of said switch means by deactivating said source of germicidal ultraviolet radiation while said door remains open; and
further controlling the activation of said source of germicidal ultraviolet radiation for a determined time interval by a timer means in said control unit.

11. The method for reducing or eliminating microbes on surfaces, equipment or vehicles in poultry processing facilities of claim 10, wherein said method further comprises
providing at least one motion detection unit,
wherein activating said motion detector unit delays or deactivates said source of germicidal ultraviolet radiation for a determined time interval.

12. The method for reducing or eliminating microbes on surfaces, equipment or vehicles in poultry processing facilities of claim 10, wherein said method further comprises
providing a manual control unit;
limiting the activation of said source of germicidal ultraviolet radiation by the manual control unit to authorized users through a key or alphanumerical pad and access code;
providing a wireless control unit capable of receiving or transmitting signals from a remote control unit;
providing a remote control unit capable of receiving or transmitting signals from a wireless control unit; and
controlling the timing intervals of said control unit for activation of said source of germicidal ultraviolet radiation by receiving or transmitting signals from a remote control unit through said wireless control unit.
